(19) United States
(12) Patent Application Publication
(10) Pub. No.: US 2025/0010089 A1
(43) Pub. Date: Jan. 9, 2025

(54) GENERATING ELECTROMAGNETIC WAVES FOR TRANSCRANIAL MAGNETIC STIMULATION

(71) Applicant: VIRGINIA TECH INTELLECTUAL PROPERTIES, INC., Blacksburg, VA (US)

(72) Inventors: Ali Hosseini-Fahraji, Blacksburg, VA (US); Majid Manteghi, Blacksburg, VA (US)

(21) Appl. No.: 18/708,683

(22) PCT Filed: Nov. 18, 2022
(86) PCT No.: PCT/US2022/080145

§ 371 Date: May 9, 2024

Related U.S. Application Data
(60) Provisional application No. 63/280,858, filed on Nov. 18, 2021.

(51) Int. Cl. A61N 2/02; A61N 2/00; A61N 2/06

(52) U.S. Cl. CPC A61N 2/02; A61N 2/006; A61N 2/06

(57) ABSTRACT

Aspects of transcranial magnetic stimulation (TMS) devices are described. In one example, the TMS device includes a magnetic flux shutter that includes a multi-turn coil and a magnetic film. The magnetic film extends in a loop to encircle a region, and the multi-turn coil is continuously wound around the magnetic film. The TMS device also includes a magnet that is positioned in the region within a boundary of the magnetic film, with the magnet generating a magnetic flux. The TMS device also includes a current source coupled to the multi-turn coil. The current source is configured to deliver a current to the multi-turn coil, to vary a permeability of the magnetic film and a density of the magnetic flux extending outside the region of the magnetic flux shutter during operation.

### CROSS REFERENCE TO RELATED APPLICATIONS

This application claims the benefit of and priority to U.S. Provisional Application Ser. No. 63/280,858, filed Nov. 18, 2021, titled “GENERATING ELECTROMAGNETIC WAVES FOR TRANSCRANIAL MAGNETIC STIMULATION,” the entire contents of which is hereby incorporated herein by reference.

### BACKGROUND

Transcranial magnetic stimulation (TMS) is a non-invasive brain stimulation technique utilized to study brain function and treat neurological and psychiatric disorders. TMS devices are composed of two main components, including a pulse source and a stimulation coil. The pulse source delivers a high-current, high-voltage pulse to the coil, which is positioned close a human head. The pulse source controls the intensity and timing of the pulses, while the stimulation coil is responsible for the spatial distribution of the induced electric field. The magnetic coil generates intense pulses that induce an electric field in the brain, which modulates neural activity. As a result, the pulse source and the shape of the stimulation coil can affect the focality of a TMS device.

### SUMMARY

Various embodiments of a transcranial magnetic stimulation (TMS) device are described. In one embodiment, the TMS device includes a magnetic flux shutter that includes a magnetic film and a multi-turn coil. The magnetic film extends in a loop to encircle a region, and the multi-turn coil is continuously wound around the magnetic film. The TMS device also includes a magnet positioned in the region within a boundary formed by the magnetic film. The TMS device also includes a current source coupled to the multi-turn coil. The current source is configured to deliver a time-varying current to the multi-turn coil. The current varies the permeability of the magnetic film and, at the same time, varies a density of the magnetic flux from the permanent magnet extending through the magnetic film and to an exterior of the TMS device during operation. The magnet can be embodied as a permanent magnet in one example.

An increase of the current delivered to the multi-turn coil can further increase the permeability of the TMS device to the magnetic flux. For example, the increase in the current delivered to the coil can vary the permeability (e.g., permeability coefficient) of the magnetic film, decreasing the permeability of the magnetic film in various cases, which can increase the focality and intensity of the magnetic flux in a direction extending outside the region of the magnetic flux shutter. Further, the magnetic film can be flexible and have a variable thickness in the loop around the region. In some cases, the TMS device can include a second magnet positioned within the boundary of the magnetic film, and the second magnet can be magnetically coupled to the first magnet by a ferromagnetic material. In some cases, the magnet can be curved, and the magnetic film can be asymmetrically shaped.

In addition, the magnet can include two ends that are coupled to the magnetic flux shutter, with each of the two ends being positioned a same distance from the multi-turn coil and the magnetic film. The magnetic film can include a lower section that is beneath edges of the two ends of the magnet. The magnetic film can also include an upper section that is above the magnet. Further, the magnetic film can include at least two side sections that are lateral to the magnet. The lower section of the magnetic film can have a thinner thickness than thicknesses of the upper section and the at least two side sections, in some cases.

In another example, a second TMS device includes a magnetic flux shutter that includes a magnetic film and a multi-turn coil. The magnetic film extends in a loop to encircle a region, and the multi-turn coil is continuously wound around the magnetic film. The second TMS device also includes a magnet positioned in the region within a boundary formed by the magnetic film. The magnetic film is asymmetrically shaped, and the magnet includes two ends that are coupled to the magnetic flux shutter. The magnetic film also includes a lower section, an upper section, and at least two side sections. The lower section is beneath edges of the two ends of the magnet, the upper section is above the magnet, and the at least two side sections are lateral to the magnet. The second TMS device also includes a current source coupled to the multi-turn coil. The current source is configured to deliver a time-varying current to the multi-turn coil. The current varies the permeability of the magnetic film and, at the same time, varies a density of the magnetic flux from the permanent magnet extending through the magnetic film and to an exterior of the second TMS device during operation.

In some cases, the magnetic film of the second TMS device can have a variable thickness in the loop around the region, and the upper section and the at least two side sections can have equal thicknesses. The lower section can have a thinner thickness than thicknesses of the upper section and the at least two side sections. Also, the magnet can include a permanent magnet in some cases.

In another example, a third TMS device includes a magnetic flux shutter that includes a magnetic film and a multi-turn coil. The magnetic film extends in a loop to encircle a region, and the multi-turn coil is continuously wound around the magnetic film. The third TMS device also includes a magnet positioned in the region within a boundary formed by the magnetic film. The magnet includes two ends that are coupled to a curved surface of the magnetic flux shutter, and the magnetic film includes a lower section, an upper section, and at least two side sections. The lower section is beneath edges of the two ends of the magnet, the upper section is above the magnet, and the at least two side sections are lateral to the magnet. The third TMS device also includes a current source coupled to the multi-turn coil. The current source is configured to deliver a time-varying current to the multi-turn coil. The current varies the permeability of the magnetic film and, at the same time, varies a density of the magnetic flux from the permanent magnet extending through the magnetic film and to an exterior of the third TMS device during operation. The magnetic film can have a variable thickness in the loop around the region, and the upper section and the at least two side sections can have equal thicknesses. Further, the magnet can include a permanent magnet in some cases.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 illustrates a side view of an exemplary transcranial magnetic stimulation (TMS) device according to various embodiments described herein.

FIG. 2 illustrates the cross-sectional view of the TMS device designated A-A in FIG. 1 according to various embodiments described herein.

FIG. 3 illustrates a magnetic field distribution chart on a brain surface, compared between the TMS device shown in FIGS. 1 and 2 and an exemplary figure-8 coil TMS device, according to various embodiments described herein.

FIG. 4 illustrates a magnetic field distribution chart at a depth of 10 mm inside a brain, compared between the TMS device shown in FIGS. 1 and 2 and an exemplary figure-8 coil TMS device, according to various embodiments described herein.

FIG. 5 illustrates a magnetic field distribution chart at a depth of 15 mm inside a brain, compared between the TMS device shown in FIGS. 1 and 2 and an exemplary figure-8 coil TMS device, according to various embodiments described herein.

FIG. 6 illustrates a magnetic field depth chart measured from a brain surface, compared between the TMS device shown in FIGS. 1 and 2 and an exemplary figure-8 coil TMS device, according to various embodiments described herein.

FIG. 7 illustrates a side view of a second TMS device according to various embodiments described herein.

FIG. 8 illustrates a side view of a third TMS device according to various embodiments described herein.

FIG. 9 illustrates a side view of a fourth TMS device according to various embodiments described herein.

### DETAILED DESCRIPTION

Transcranial magnetic stimulation (TMS) pulse waveforms can influence both the technical performance of TMS devices, such as the energy efficiency, heating, and selectivity of repetitive TMS, and the physiologic response to stimulation, such as the neural response latency and contralateral silent period. Some pulses are easier to generate than others, while others use less energy, resulting in less coil heating. On the other hand, different neuron types have distinct channel expression, geometry, and anatomic surroundings, resulting in distinct dynamics. TMS pulse waveforms can interact with neuron dynamics, thereby potentially influencing neural response.

However, designing a practical, flexible, and efficient pulse source is a technical challenge, since conventional TMS devices can require relatively high-energy pulses. Conventional TMS devices can require approximately 100 Joules, for example, of energy at the neural activation threshold. This energy may need to be delivered to the stimulation coil in less than a tenth of a millisecond. Additionally, due to the poor electromagnetic coupling between the coil and the neurons, only a small portion of the energy may be transferred to the target neurons. The majority of the energy often remains in the stimulator circuit or is converted to heat. As a result, the source may need to deliver a high-energy pulse to the coil with peak voltages in the kilovolt range (e.g., such as up to 3 kV), peak currents in the kiloampere range (e.g., such as up to 10 kA), and pulse widths ranging from 100 s to 1 ms. The power consumption of conventional TMS devices is also directly proportional to the pulse repetition rate. At maximum pulse strength, the pulse repetition rate in repetitive TMS (rTMS) devices can reach 100 Hz, resulting in power consumption of up to 10 kW.

In many conventional TMS devices, the pulse waveform is produced by an energy oscillation between the storage capacitors and the stimulation coil. The storage capacitance, coil inductance, and circuit resistance can all influence the oscillation period of the pulse waveform. The most common pulse source waveforms are biphasic and monophasic. The biphasic waveforms involve the positive and negative electric field phases with similar amplitude. In contrast, the monophasic waveforms produce a unidirectional (asymmetric) electric field, which causes a fast magnetic field (current) rise time and a slow fall time. However, due to high current pulses, these sources offer a limited selection of pulse shapes that are largely determined by the ease of generating sinusoidal pulses with fixed durations rather than paying attention to physiological or electrical efficiency. Although other attempts have been made to address some of these limitations, they still face different challenges, especially providing efficient pulse energy recycling. Hence, commercially available TMS devices lack flexible pulse shape and intensity control to improve neuromodulation performance and target desired neural populations.

The locus of neural activation in TMS applications can be determined by the induced field distribution. The induced field distribution can be affected by the stimulating coil geometry and placement. The stimulation locus in the brain is roughly in the region of the brain where the electric field is strongest. Electric currents are induced where the coil is tangential to the scalp, whereas non-tangential coil elements give rise to a surface charge that can cancel the induced field and cause rapid field dissipation. As a result, various coils for TMS devices have been evaluated or proposed to optimize field distribution. Generally, focality can be enhanced by reducing the diameter of the coil windings, as smaller coils can be more focal due to improved confinement of magnetic flux. However, smaller coils exhibit greater attenuation of the electric field with distance and have limited feasibility because of the high forces and heating that occur within a small volume.

Many commercial TMS devices utilize two types of coils, particularly circular and figure-8 coils. Circular coils induce a non-focal ring-shaped electric field maximum, potentially stimulating brain regions under the coil perimeter. In contrast, the figure-8 coil includes a pair of adjacent circular loops with current flowing in opposite directions, producing a relatively focal electric field maximum under the center of the coil where the two loops meet. Figure-8 coils generally have better focality than circular coils.

Several attempts have been made to increase the focality of TMS coils in TMS devices. For example, circular coils have been modified with an angulated extension or with a variation of the winding density or concavity to focalize the stimulation. However, none of these approaches significantly improves the focality of circular coils. For figure-8 coils, cloverleaf (e.g., four sets of nearly circular windings), slinky (e.g., multiple circular or rectangular loops connected together at one edge and fanned out to from a half toroid), and eccentric or 3-D array coil designs have been implemented. These coils have better focality than standard figure-8 coils with similar outer dimensions, but they face challenges such as requirement of sophisticated manufacturing processes and cooling systems.

Additionally, attempts have been made to increase stimulation depth to directly stimulate deep brain structures. Larger coils, in general, induce a more deeply penetrating and less focal electric field than smaller ones, which may increase the risk of seizures and other side effects. Some coils that are currently available for deeper penetration have more complex winding patterns and larger dimensions than conventional TMS coils, resulting in slower electric field attenuation with depth at the expense of reduced focality. In sum, existing TMS devices have limitations in targeting desired neural populations, due to depth-focality tradeoffs and reliance on high-energy pulse sources.

To address the limitations outlined above, aspects of the embodiments described herein include a transcranial magnetic stimulation (TMS) device that modulates a magnetic flux Φ_{P} of a permanent magnet to electrically stimulate neurons of a human or other patient during operation. The TMS device in accordance with the embodiments described herein includes a magnetic flux shutter that includes a multi-turn coil and a magnetic film. The magnetic film extends in a loop to encircle a region, and the multi-turn coil is continuously wound around the magnetic film. The TMS device also includes a magnet that is positioned in the region within a boundary of the magnetic film, with the magnet generating a magnetic flux Φ_{P}. Further, the TMS device also includes a current source coupled to the multi-turn coil. The current source is configured to deliver a current to the multi-turn coil, to vary a permeability of the magnetic film and a density of the magnetic flux Φ_{P} extending outside the region of the magnetic flux shutter during operation. The magnetic film can be asymmetrically shaped and vary in thickness and in width in various embodiments. In some examples, the magnetic film can have a lower section, an upper section, and at least two side sections.

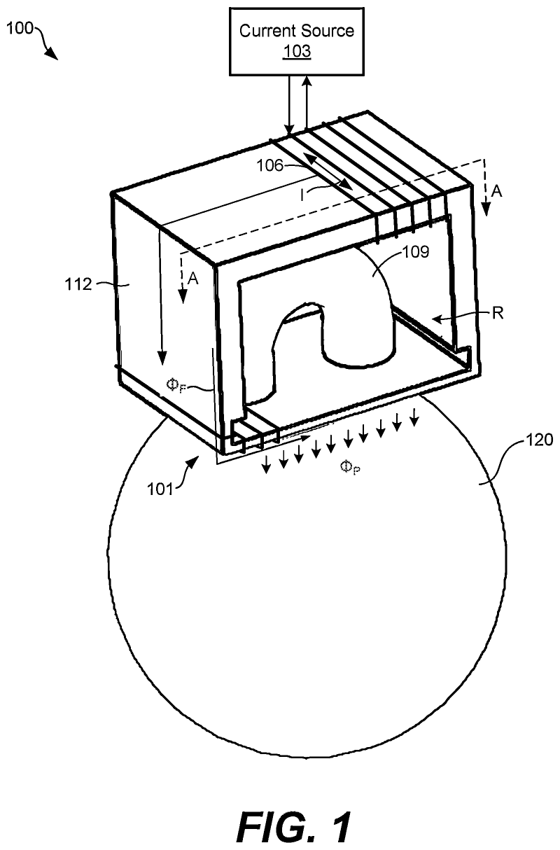

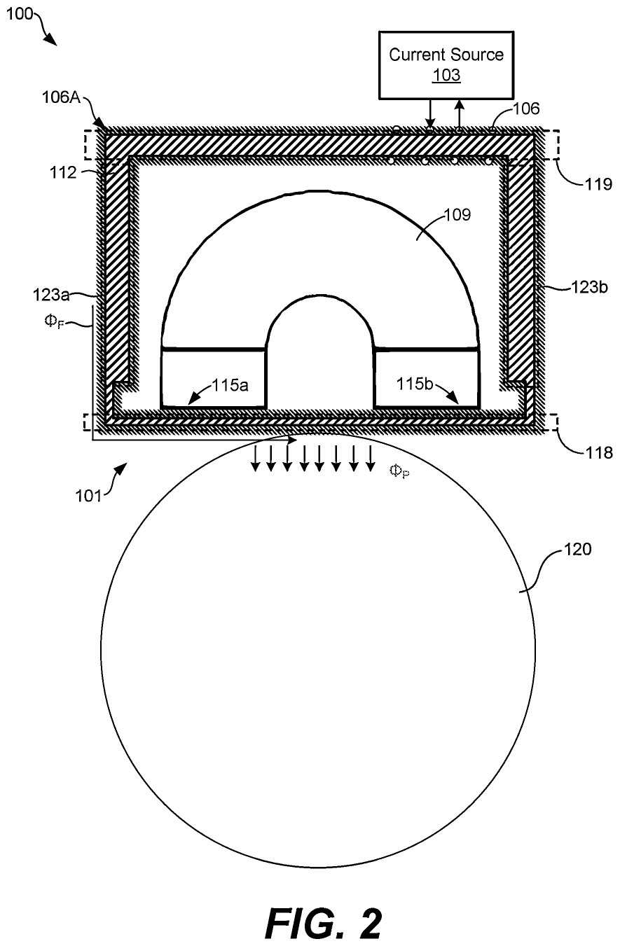

Referring now to the drawings, FIG. 1 illustrates an exemplary TMS device 100 according to various embodiments described herein. FIG. 2 illustrates the cross-sectional view of the TMS device designated A-A in FIG. 1. The TMS device 100 is a representative example of a device that can be utilized for generating electromagnetic waves for transcranial magnetic stimulation, as described herein. The TMS device 100 is not drawn to any particular size or scale, and the shape and size of the TMS devices described herein can vary as compared to those shown. The TMS device 100 is also not exhaustively illustrated in FIGS. 1 and 2, and the TMS device 100 can include other components although not shown. Alternatively, one or more of the components of the TMS device 100 shown in FIG. 1 can be omitted in some cases.

The TMS device 100 includes a current source 103, a multi-turn coil 106 (“coil 106”), a magnetic film 112, and a magnet 109. The coil 106 and the magnetic film 112, arranged together, form a type of magnetic flux shutter. The magnetic film 112 extends in a loop and encircles a region “R.” The coil 106 is continuously wound around the magnetic film 112. The magnet 109 is positioned in the region “R,” within (i.e., encircled by) a boundary formed by the magnetic flux shutter formed by the magnetic film 112 and the multi-turn coil 106. In operation, a treatment region or surface 101 of the TMS device 100 can be placed proximate to a scalp 120 of an individual or patient for treatment.

The coil 106 can be embodied as a continuous length of wire. The coil 106 is continuously wound around the magnetic film 112 in the direction shown in FIG. 1. The wire of the coil 106 extends in a direction that is substantially perpendicular to the extension of the magnetic film 112 for enclosure of the region “R.” Free wire ends of the coil 106 are electrically connected or coupled to the current source 103, such that the current source 103 can feed a current “I” through the coil 106. Based on the flow of the current “I,” a magnetic flux Φ_{F} can be induced in the magnetic film 112, as described herein. The designation of the magnetic flux Φ_{F} is shown outside the magnetic film 112 in FIGS. 1 and 2, but the magnetic flux Φ_{F} is flux extending within and along the magnetic film 112.

The turns of the coil 106 are representative in FIGS. 1 and 2. In practice, the number and spacing of the turns of the wire for the coil 106 can vary. The entire coil 106 is not illustrated in FIG. 1, for simplicity, but the coil 106 is wrapped around the magnetic film 112 along all sides that encircle the magnet 109. In FIG. 2, the region 106A designates an example of the area in which the turns of the coil 106 may extend around the magnetic film 112, in various embodiments.

The coil 106 winds around the magnetic film 112 a number of times. The coil 106 induces the magnetic flux Φ_{F} in the magnetic film 112, when the current “I” from the current source 103 flows through the coil 106. The coil 106 can be wound between 50 and 100 times around the magnetic film 112, as examples, although other numbers of turns or windings of the coil 106 can be relied upon. The number of turns of the coil 106 can depend on a combination of design factors, including the permeability of the magnetic film 112, the magnetic flux generated by the magnet 109, the impedance of the current source 103, and other factors. These and other design factors also determine the magnetic field and flux strength, density, and direction provided by the TMS device 100 overtime, as described herein.

The coil 106 can be embodied as a solid or braided copper wire, although other conductive wires can be used, such as wire formed of silver, aluminum, or other conductive materials. In one example, the coil 106 can be embodied as enameled copper wire (e.g., magnet wire) of a suitable gauge based on the number of turns in the coil 106, the current handling capacity of the coil 106, and other factors. Example gauges include 26, 24, 22, or 20 AWG wire, although other gauges of wire can be relied upon. The coil 106 can also be embodied as wire having aplastic or polymer sheath, rather than an enamel coating, braided (e.g., Litz) wire, or other types of wire.

The magnetic film 112 can be embodied as one or more layers of film formed from a cobalt, iron, nickel, certain rare-earth metals, or other magnetic material or materials. In one example, the magnetic film 112 can be embodied as one or more layers of the METGLAS® 2705M Alloy film, although other magnetic films can be relied upon. Additional aspects of the magnetic film 112 are described below. The number of layers of the magnetic film 112 can vary around the magnetic shutter. For example, the magnetic film 112 can include fewer layers in the treatment region 101 of the TMS device 100 and more layers outside the treatment region 101.

The magnet 109 can include one or more permanent magnets, such as alnico, samarium cobalt, ferrite, neodymium, or other types of permanent magnets. In some cases, the magnet 109 can be embodied as particles of alnico, samarium cobalt, ferrite, neodymium, or other magnetic material mixed with a flexible rubber binder. In other cases, the magnet 109 can include an electromagnet or a combination of an electromagnet and a permanent magnet. The magnet 109 generates a magnetic flux Φ_{P}. The magnet 109 is positioned and arranged within the region “R,” such that field lines associated with the magnetic flux Φ_{P} extend in a direction substantially perpendicular to a surface of the magnetic film 112.

The current source 103 can be embodied as a source of current. The current source 103 is configured to generate a time-varying current in preferred embodiments. As examples, the current source 103 can generate a current waveform that is sinusoidal, triangular, or square in waveform shape, at any suitable frequency or frequencies for TMS. The current source 103 can be configured to generate a range of different TMS pulse waveforms, as the use of different waveforms can influence the performance of the TMS device 100 for a range of therapeutic applications. The different waveforms can result in a range of physiologic responses to the time-varying magnetic fields generated by the TMS device 100. The range of waveforms control the TMS device 100 for a range of physiologic responses from different neurons, each of which can have a distinct response dynamic.

The TMS device 100 can be positioned as close as possible to the scalp 120 of an individual or patient, to increase performance of the TMS device 100, during use. During operation, the TMS device 100 is positioned proximally and/or tangentially to the scalp 120, as shown in FIGS. 1 and 2. The TMS device 100 can be relied upon to electrically stimulate neurons (e.g., of a human) during operation based on a modulated magnetic flux Φ_{P} of the magnet 109 according to the design of the TMS device 100.

According to aspects of the embodiments described herein, the otherwise static magnetic flux Φ_{P} generated by the magnet 109 is modulated by changing the reluctance (permeability) of the magnetic film 112. The reluctance (permeability) of the magnetic film 112 in the magnetic flux shutter varies over time based on the flow of current “I” from the current source 103. The magnitude and direction of the magnetic flux Φ_{P} of the TMS device 100 is thus made to be time-variant based on the flow of current “I,” as it alters the saturation and permeability of the magnetic film 112 surrounding the magnet 109.

Magnetic domains in the magnetic film 112 behave in part as small permanent magnets. Without the presence of an external magnetic field, the magnetic domains in the magnetic film 112 can be randomized or scattered, without being aligned. The magnetic domains in the magnetic film 112 are directed, however, by two fields in the TMS device 100. Particularly, the magnetic domains in the magnetic film 112 can be directed and aligned by the magnetic field generated by the magnet 109 and also by the magnetic field generated by the coil 106, when the current source 103 generates the current “I” in the coil 106. The fields generated by the magnet 109 and the coil 106 have different directions based on the structural design of the TMS device 100.

When a magnetizing field is applied to the magnetic film 112 by only the magnet 109 (i.e., and not by the coil 106), the field aligns the magnetic domains in the magnetic film 112 in a first direction. Depending on the field strength of the magnet 109 and the extent of the magnetic domains in the magnetic film 112, the magnetic domains in certain areas or regions of the magnetic film 112 are aligned to an extent in the first direction, possibly leading to saturation in those areas or regions if the magnetic domains are fully aligned to the extent possible. On the other hand, when a magnetizing field is applied to the magnetic film 112 by only the coil 106 (i.e., and not by the magnet 109), the field aligns the magnetic domains in the magnetic film 112 in a second direction, different than the first direction. Depending on the field strength provided by the coil 106, the magnetic domains in certain areas or regions of the magnetic film 112 are aligned to an extent in the second direction, possibly leading to saturation in those areas or regions if the magnetic domains are fully aligned to the extent possible. Overall, the magnetic permeability p of the magnetic film 112 varies based on the magnetic flex density B in the magnetic film 112 and the strength of the magnetic field H acting on the magnetic film 112, as is understood in the field.

Given that the field strength and magnetic flux Φ_{P} of the magnet 109 is constant, the current “I” delivered to the coil 106 by the current source 103 results in a variation of the permeability of the magnetic film 112. This variation results in the TMS device 100 operating between two modes or states, including “open mode” and “closed mode” states, as well as regions of operation between the open and closed modes. During operation of the TMS device 100 with a time-varying current provided by the current source 103, the static magnetic flux Φ_{P} of the magnet 109 is, at least in part, blocked or diverted from extending outside the TMS device 100 in closed mode operation. When no (or relatively low) current is delivered to the coil 106 by the current source 103, the permeability of the magnetic film decreases, and the TMS device 100 operates open mode. The magnetic flux Φ_{P} of the magnet 109 extends beyond or outside the TMS device 100 to a greater extent in open mode as compared to closed mode. A more detailed explanation of these two modes, along with the structure of the TMS device 100 is discussed with respect to the cross-sectional view shown in FIG. 2.

FIG. 2 illustrates a cross-sectional view of the TMS device 100 according to various embodiments described herein. As shown, the magnetic film 112 surrounds the magnet 109 by forming a boundary around the magnet 109. As noted above, the magnetic film 112 can include one or more layers of film formed of ferromagnetic materials, such as cobalt, iron, nickel, certain rare-earth metals, and other ferromagnetic materials. In one example, the magnetic film 112 can be embodied as one or more layers of the METGLAS® 2705M Alloy film, although other magnetic films can be relied upon. In some cases, the magnetic film 112 can include a soft and a relatively flexible or pliable magnetic material that can accommodate a wide variety of shapes and sizes.

In the example shown in FIGS. 1 and 2, the magnetic film 112 extends in a continuous loop and surrounds the magnet 109, by forming a rectangular boundary around the magnet 109. Although a rectangular boundary is shown in FIGS. 1 and 2, the magnetic film 112 can be flexed into various sizes and shapes to accommodate a desired shape (e.g., cubical, spherical, etc.) of the TMS device 100 based on the target application.

The permanent magnet 109 can operate as a source of the magnetic flux Φ_{P}, and the magnetic film 112 can operate as a fluxgate of the magnetic flux Φ_{P} as described herein. The electric current “I” from the current source 103 can control, in part, the permeability of the magnetic film 112, which acts as a current-controlled shutter for the magnetic flux Φ_{P}. When no current is delivered to the coil 106, for example, a first amount or extent of the magnetic flux Φ_{P} of the magnet 109 passes beyond the magnetic film 112. When current is delivered to the coil 106, the permeability of the magnetic film 112 varies due to the addition of the magnetic flux Φ_{F} generated by the coil 106. A second amount or extent of the magnetic flux Φ_{P} of the magnet 109 passes beyond the magnetic film 112 in this case. As the electric current “I” from the current source 103 varies over time, the amount or extent of the magnetic flux Φ_{P} of the magnet 109 that extends beyond the treatment region or surface 101 of the TMS device 100 also varies.

Thickness of the magnetic film 112 can play an important role in the performance of the TMS device 100. In one example, the magnetic film 112 should be thick enough to block the magnetic flux Φ_{P} of the magnet 109 in the closed mode, while remaining thin enough to allow the magnetic flux Φ_{P} to flow through the magnetic film 112 in the open mode. Additionally, the magnetic flux that flows through the magnetic film 112 in the open mode should ideally occur only in the direction proximal to the scalp 120 for increased performance and efficiency of the TMS device 100. For example, the TMS device 100 can achieve a higher modulation depth, intensity, and focality in the direction proximal to (i.e., directed into) the scalp 120 and indicated by the magnetic flux Φ_{P} arrows, while reducing or preventing the magnetic flux Φ_{P} from escaping upper and side sections of the magnetic film 112.

In order to achieve the higher modulation depth, intensity, and focality of the magnetic flux Φ_{P} in the direction proximal to the scalp 120, the magnetic film 112 can vary in thickness based on positional proximity to the scalp 120. A lower section of the magnetic film 112 that is positioned closer or proximal to the scalp 120 can have a thinner thickness than an upper section of the magnetic film 112 that is positioned distal to the scalp 120 or further away from the scalp 120. Further, side sections of the magnetic film 112 that are lateral to the magnet 109 can be thicker than the lower section to function as a shielding layer.

In the example shown in FIG. 2, a lower section 118 of the magnetic film 112 that is beneath edges 115a and 115b of the magnet 109 and proximal to the scalp 120 is thinner than an upper section 119 of the magnetic film 112 that is above the magnet 109 and distal to the scalp 120. In addition, the lower section 118 has a thinner thickness than a thicknesses of side sections 123a and 123b that are lateral to the position of the magnet 109. In various embodiments, the upper section 119 and the side sections 123a and 123b can have equal thicknesses or substantially equal thicknesses. The upper section 119 and the side sections 123a and 123b can be formed using a first number of layers of magnetic film, and the lower section 118 can be formed using a second number of layers of magnetic film. The first number of layers can be greater than the second number of layers. In some cases, magnetic film 112 can be embodied as several laminated layers of magnetic film, and the upper section 119 and the side sections 123a and 123b can be formed using a first number of laminated layers of magnetic film, and the lower section 118 can be formed using a second number of laminated layers of magnetic film.

The upper section 119 and the side sections 123a and 123b function as shielding layers to reduce or prevent the magnetic field generated by the magnet 109 from escaping laterally and above the magnet 109 in the TMS device 100. The shielding layers provide increased efficiency for the TMS device 100, by minimizing power dissipated due to heat, while enabling increased magnetic flux density to flow through the lower section 118 and to penetrate into the scalp 120. Additionally, the magnetic film 112 can be asymmetrically shaped to mitigate a compromise that can exist between a size of the magnet 109 and maximum leakage field induced based on the size.

The magnet 109 is also positioned as close as possible to the scalp 120 while making contact with the coil 106 and staying within the boundary formed by the magnetic film 112. According to one example, the two edges 115a and 115b of the magnet 109 are each positioned with respect to the coil 106 so that there is an equal separation distance from the lower section 118 of the magnetic film 112 to the two edges 115a and 115b.

According to an experiment conducted involving the TMS device 100, the magnet 109 included a N52 permanent magnet, and the magnetic film 112 included a Metglas® film with variable thickness. The coil 106 included a 75-turn control coil that was wrapped around the Metglas® film. The magnet 109 included a U-shape permanent magnet to compare against the overall performance of a figure-8 coil, since a figure-8 coil is generally more focal than other types of coils. The variable thickness of the Metglas® film aided in switching system operation between the open and closed modes.

In a magnetostatic simulator, the performance of the TMS device 100 was compared to that of a 70 mm figure-8 coil. The induced magnetic field distribution inside a spherical head model was computed, and critical parameters such as focality and depth of field penetration were compared. For this experiment, the 70 mm figure-8 coil was bent proportionally to create a strong magnetic, field and was excited by a 5000 A-turn current, which directly controlled the generated magnetic field strength. For the TMS device 100, a 75 A-turn current was used to change or vary the modes of operation and the size of the magnet 109 played an important role in terms of the magnetic field strength. The type and size of the magnet 109 was chosen appropriately to produce a field close to that of the figure-8 coil. An advantage of the TMS de % vice 100 over the figure-8 coil is that the penetrated field is determined by the volume and shape of the magnet 109 rather than the coil current. Therefore, there is less concern about safety or local discomfort to the scalp. Furthermore, using less current to modulate the coil 106, as compared to that of the figure-8 coil, provided more control on the intensity, temporal, and timing of the pulses so that overall TMS performance was be improved.

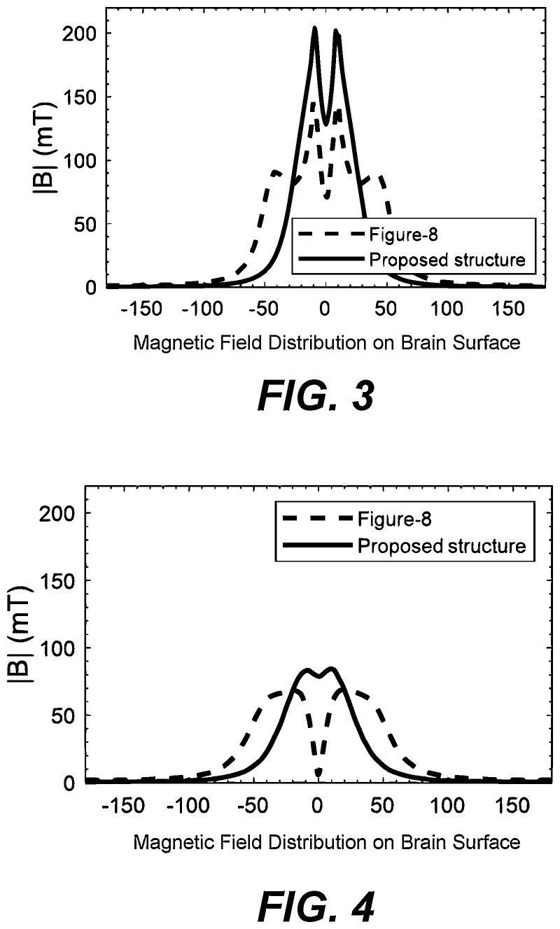

FIG. 3 lustrates a magnetic field distribution chart on a brain surface, compared between the TMS device 100 shown in FIGS. 1 and 2 and the exemplary figure-8 coil TMS device described above, according to various embodiments described herein. FIG. 3 illustrates that the decay of the magnetic field to half of its maximum value on the brain surface for the TMS device 100 is limited to ±30°, whereas it is around ±50° for the figure-8 coil.

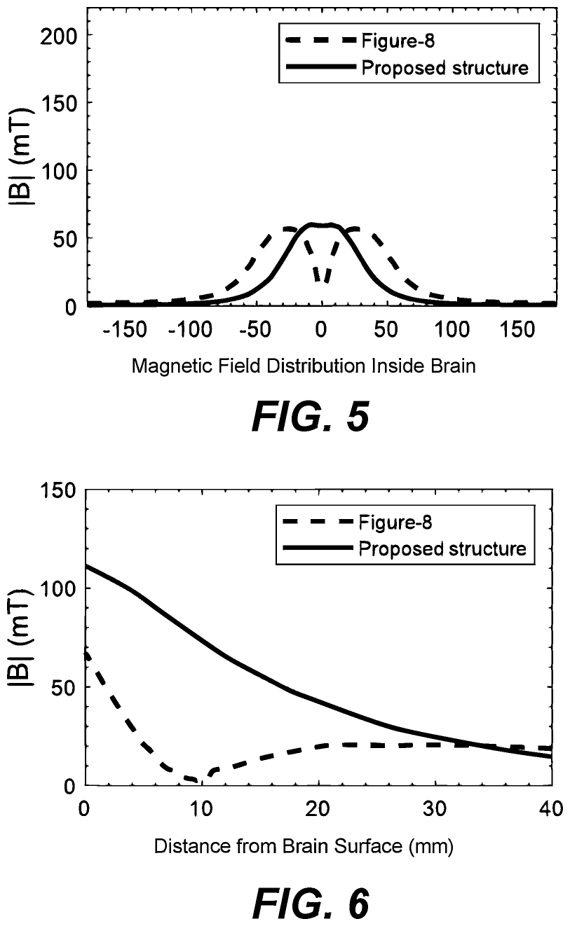

FIGS. 4 and 5 illustrate a magnetic field distribution chart at a depth of 10 mm and 15 mm inside a brain, respectively, compared between the TMS device 100 shown in FIGS. 1 and 2 and the exemplary figure-8 coil TMS device described above, according to various embodiments described herein. In FIGS. 3-5, the TMS device 100 exhibits increased focality compared to that of the exemplary figure-8 coil TMS device.

FIG. 6 illustrates a magnetic field depth chart measured from a brain surface, compared between the TMS device 100 shown in FIGS. 1 and 2 and the exemplary figure-8 coil TMS device described above, according to various embodiments described herein. FIG. 6 illustrates that the TMS device 100 can stimulate neurons inside the brain with a stronger field, demonstrating that the penetration depth of the induced field is improved and consumes significantly less input current.

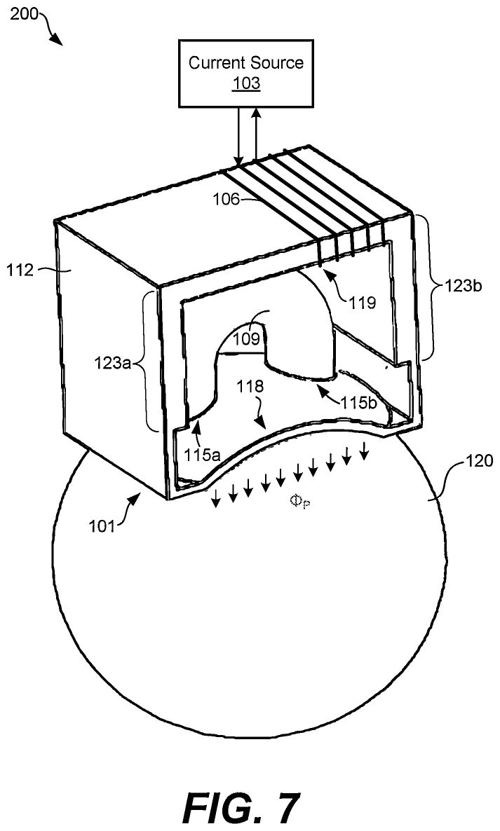

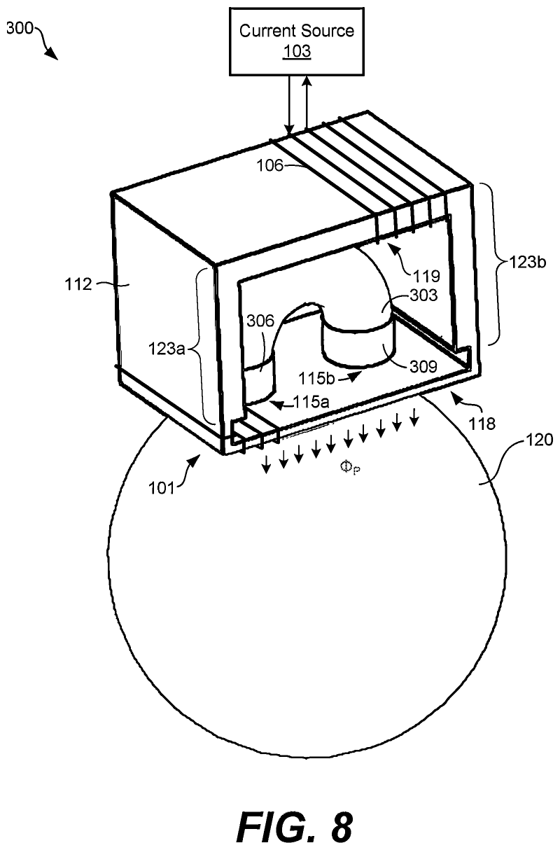

FIG. 7 illustrates a second TMS device 200 according to various embodiments described herein. The TMS device 200 includes many of the same components described with respect to the TMS device 100. For example, the TMS device 200 includes the multi-turn coil 106 that continuously winds around the magnetic film 112, the magnet 109, and the current source 103. The magnet 109 is positioned within a boundary formed by the magnetic film 112 and the multi-turn coil 106 that winds around the magnetic film 112. The multi-turn coil 106 and the magnetic film 112 are configured to be positioned proximally to the scalp 120 to electrically stimulate neurons during operation based on a modulated magnetic flux Φ_{P} of the magnet 109. The modulation of the magnetic flux Φ_{P} of the magnet 109 can occur based on a current delivered from the current source 103, which can alter the permeability of the magnetic film 112.

Similar to the TMS device 100, the magnetic film 112 of the TMS device 200 is flexible and can vary in thickness. In the TMS device 200, the lower section 118 of the magnetic film 112, which is beneath the edges 115a and 115b and proximal to the scalp 120, has a curved shape to better accommodate the curve of the scalp 120. The lower section 118 can have a thinner thickness than a thicknesses of the upper section 119 and the side sections 123a and 123b, so that a magnetic flux Φ_{P} leakage of the magnet 109 is increased through the lower section 118. The upper section 119 and the side sections 123a and 123b can function as shielding layers to prevent magnetic flux Φ_{P} from escaping laterally and above the magnet 109. In various embodiments, the upper section 119 and the side sections 123a and 123b can have equal thicknesses. In comparison to that of the TMS device 100, the TMS device 200 can achieve increased magnetic flux Φ_{P} focality that is delivered through the scalp 120. However, despite the increased focality, magnetic field penetration strength and depth may suffer in comparison to that of the TMS device 100.

FIG. 8 illustrates a third TMS device 300 according to various embodiments described herein. The TMS device 300 includes many of the same components described above with respect to the TMS device 100. For example, the TMS device 300 includes the multi-turn coil 106 that continuously winds around the magnetic film 112 and the current source 103. However, the TMS device 300 includes two separate magnets 306 and 309 that are coupled together by a ferromagnetic material 303. The ferromagnetic material 303 may be soft and flexible, like the magnetic film 112, and can be flexed or bent into a curved shape as depicted. In some cases, however, the ferromagnetic material 303 may be flexed into a different shape (e.g., rectangular, cubical, etc.). In comparison with the TMS device 100, the inclusion of the two separate magnets 306 and 309 coupled together by the ferromagnetic material 303 allows reduction of device size while maintaining device performance for the TMS device 300.

The magnets 306 and 309 can include permanent magnets, such as alnico, samarium cobalt, ferrite, flexible rubber, neodymium magnets, and other types of permanent magnets. In some cases, the magnets 306 and 309 may include electromagnets or a combination of electromagnets and permanent magnets. Similar to the magnet 109 in the TMS devices 100 and 200, the magnets 306 and 309 are positioned within a boundary formed by the magnetic film 112 and the multi-turn coil 106. Further, the magnetic film 112 surrounds the magnets 306 and 309. Additionally, the ferromagnetic material 303 enables the magnets 306 and 309 to remain in specific positions, while reducing the overall size of the TMS device 300. The multi-turn coil 106 and the magnetic film 112 are configured to be positioned proximally to the scalp 120 to electrically stimulate neurons during operation based on a modulated magnetic flux Φ_{P} of the magnets 306 and 309. The modulation of the magnetic flux Φ_{P} of the magnets 306 and 309 can occur based on a current delivered from the current source 103, which can alter the permeability of the magnetic film 112 to the magnetic flux Φ_{P} of the magnets 306 and 309.

As discussed above with respect to the TMS devices 100 and 200, the magnetic film 112 is flexible and can vary in thickness. In the TMS device 300, the lower section 118 of the magnetic film 112, which is beneath the edges 115a and 115b and proximal to the scalp 120, has a rectangular shape. The lower section 118 can have a thinner thickness than the thicknesses of the upper section 119 and the side sections 123a and 123b, so that a magnetic flux Φ_{P} leakage of the magnets 306 and 309 is increased through the lower section 118. The upper section 119 and the side sections 123a and 123b can function as shielding layers, to reduce or prevent the magnetic field from escaping laterally and above the magnets 306 and 309. In various embodiments, the upper section 119 and the side sections 123a and 123b can have equal thicknesses. In comparison to the TMS device 100, the TMS device 300 can offer reduced device size while maintaining performance. However, efficiency may decrease in some cases, due to loss in the magnetic material 303.

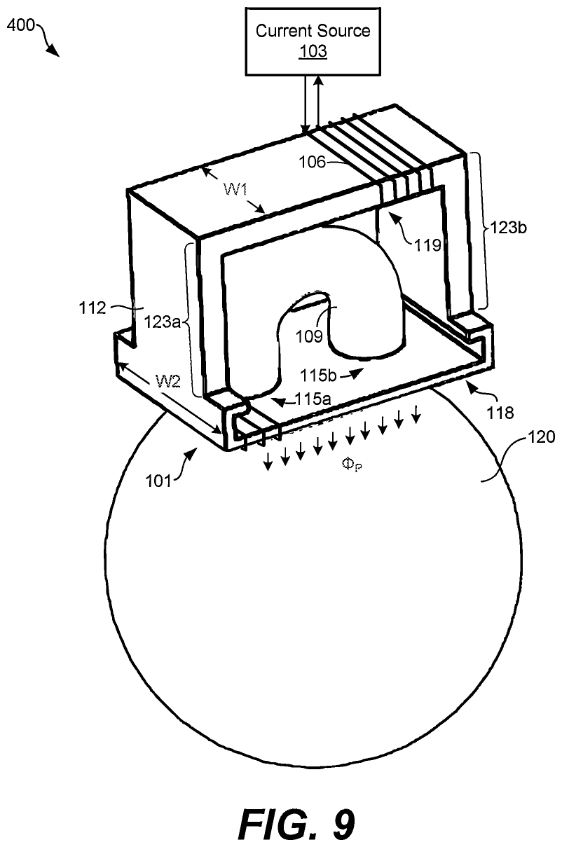

FIG. 9 illustrates a fourth TMS device 400 according to various embodiments described herein. The TMS device 400 includes many of the same components described with respect to the TMS device 100. For example, the TMS device 400 includes the multi-turn coil 106 that continuously winds around the magnetic film 112, the magnet 109, and the current source 103. The magnet 109 is positioned within a boundary formed by the magnetic film 112 and the multi-turn coil 106 that winds around the magnetic film 112. The multi-turn coil 106 and the magnetic film 112 are configured to be positioned proximally to the scalp 120 to electrically stimulate neurons during operation based on a modulated magnetic flux Φ_{P} of the magnet 109. The modulation of the magnetic flux Φ_{P} of the magnet 109 can occur based on a current delivered from the current source 103, which can alter the permeability of the magnetic film 112.

Similar to the TMS device 100, the magnetic film 112 of the TMS device 400 is flexible and can vary in thickness. In the TMS device 400, the lower section 118 of the magnetic film 112 is beneath the edges 115a and 115b and proximal to the scalp 120. The lower section 118 can have a thinner thickness than a thicknesses of the upper section 119 and the side sections 123a and 123b, so that a magnetic flux Φ_{P} leakage of the magnet 109 is increased through the lower section 118. The upper section 119 and the side sections 123a and 123b can function as shielding layers to prevent magnetic flux Φ_{P} from escaping laterally and above the magnet 109. In various embodiments, the upper section 119 and the side sections 123a and 123b can have equal thicknesses.

The TMS device 400 includes differing widths W1 and W2, with W1 corresponding to a width of the upper section 119 and the side sections 123a and 123b, and W2 corresponding to a width of the lower section 118. The width W1 is smaller or narrower than the width W2, and the width W1 is narrower than a width corresponding to the upper section 119, the side sections 123a and 123b, and the lower section 118 of the TMS device 100. Incorporation of the narrower width W1 results in overall structure volume reduction for the TMS device 400 compared to that of the TMS device 100. In various cases, the incorporation of the narrower width W1 may contribute to the increase of the magnetic flux leakage Φ_{P} of the magnet 109 through the lower section 118 of the magnetic film 112 for the TMS device 400. In comparison to the TMS device 100, the TMS device 400 can achieve similar focality and magnetic field penetration depth, while benefiting from lower power dissipation and lower overall cost due to the reduced structure volume.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The features, structures, and components described herein may be combined in one or more embodiments in any suitable manner, and the features discussed in the various embodiments are interchangeable in many cases. When two components are described as being “coupled to” each other, the components can be electrically coupled to each other, with or without other components being electrically coupled and intervening between them. When two components are described as being “directly coupled to” each other, the components can be electrically coupled to each other, without other components being electrically coupled between them.

Terms such as “a,” “an,” “the,” and “said” are used to indicate the presence of one or more elements and components. The terms “comprise,” “include,” “have,” “contain,” and their variants are used to be open ended and may include or encompass additional elements, components, etc., in addition to the listed elements, components, etc., unless otherwise specified. The terms “first,” “second,” etc. are used only as labels, rather than a limitation for a number of the objects.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, at least the following is claimed:

1. A transcranial magnetic stimulation (TMS) device, comprising:
a magnetic flux shutter comprising a magnetic film and a multi-turn coil, the magnetic film extending in a loop to encircle a region and the multi-turn coil being continuously wound around the magnetic film;
a magnet positioned in the region within a boundary of the magnetic film, the magnet generating a magnetic flux; and
a current source coupled to the multi-turn coil, the current source being configured to deliver a current to the multi-turn coil, to vary a permeability of the magnetic film and a density of the magnetic flux extending outside the region of the magnetic flux shutter during operation.

2. The TMS device of claim 1, wherein the magnet comprises a permanent magnet.

3. The TMS device of claim 1, wherein an increase of the current delivered to the multi-turn coil decreases the permeability of the magnetic film, the decrease in the permeability increasing focality and intensity of the magnetic flux in a direction extending outside the region of the magnetic flux shutter.

4. The TMS device of claim 1, wherein the magnetic film is flexible.

5. The TMS device of claim 1, wherein the magnetic film comprises a variable thickness in the loop around the region.

6. The TMS device of claim 1, further comprising a second magnet positioned within the boundary of the magnetic film, the second magnet being magnetically coupled to the magnet by a ferromagnetic material.

7. The TMS device of claim 1, wherein the magnet is curved.

8. The TMS device of claim 1, wherein the magnetic film is asymmetrically shaped.

9. The TMS device of claim 1, wherein the magnet comprises two ends, each of the two ends being positioned a same distance from the coil and the magnetic film.

10. The TMS device of claim 9, wherein the magnetic film comprises:
a lower section beneath edges of the two ends of the magnet;
an upper section above the magnet; and
at least two side sections lateral to the magnet.

11. The TMS device of claim 10, wherein the lower section of the magnetic film comprises a thinner thickness than thicknesses of the upper section and the at least two side sections.

12. A transcranial magnetic stimulation (TMS) device, comprising:
a magnetic flux shutter comprising a magnetic film and a multi-turn coil, the magnetic film extending in a loop to encircle a region and the multi-turn coil being continuously wound around the magnetic film, the magnetic film being asymmetrically shaped;
a magnet that is positioned in the region within a boundary of the magnetic film, the magnet generating a magnetic flux, the magnet comprising two ends that are coupled to the magnetic flux shutter,
wherein the magnetic film comprises:
a lower section beneath edges of the two ends of the magnet;
an upper section above the magnet; and
at least two side sections lateral to the magnet; and
a current source coupled to the multi-turn coil, the current source being configured to deliver a current to the multi-turn coil, to vary a permeability of the magnetic film and a density of the magnetic flux extending outside the region of the magnetic flux shutter during operation.

13. The TMS device of claim 12, wherein the magnetic film comprises a variable thickness in the loop around the region.

14. The TMS device of claim 12, wherein the upper section and the at least two side sections are equal in thickness.

15. The TMS device of claim 12, wherein the lower section comprises a thinner thickness than thicknesses of the upper section and the at least two side sections.

16. The TMS device of claim 12, wherein the magnet comprises a permanent magnet.

17. A transcranial magnetic stimulation (TMS) device, comprising:
a magnetic flux shutter comprising a magnetic film and a multi-turn coil, the magnetic film extending in a loop to encircle a region and the multi-turn coil being continuously wound around the magnetic film;
a magnet that is positioned in the region within a boundary of the magnetic film, the magnet generating a magnetic flux, the magnet comprising two ends that are coupled to a curved surface of the magnetic flux shutter,
wherein the magnetic film comprises:
a lower section beneath edges of the two ends of the magnet;
an upper section above the magnet; and
at least two side sections lateral to the magnet; and
a current source coupled to the multi-turn coil, the current source being configured to deliver a current to the multi-turn coil, to vary a permeability of the magnetic film and a density of the magnetic flux extending outside the region of the magnetic flux shutter during operation.

18. The TMS device of claim 17, wherein the magnetic film comprises a variable thickness in the loop around the region.

19. The TMS device of claim 17, wherein the upper section and the at least two side sections comprise equal thicknesses.

20. The TMS device of claim 17, wherein the magnet comprises a permanent magnet.
